(19) United States
(12) Patent Application Publication
(10) Pub. No.: US 2003/0059337 A1
(43) Pub. Date: Mar. 27, 2003

(54) Method for the preservation of paper products

(76) Inventors: Antonio Zappala', Rome (IT); Andrea Gorassini, Udine (IT); Angelo Guimanini, Udine (IT); Giancarlo Verardo, Udine (IT)

Correspondence Address: Jay S. CINAMON, ABELMAN, FRAYNE & schwab, Attorneys at Law, 150 East 42nd Street, New York, NY, 10017-5612, US

(21) Appl. No.: 10/189,694

(22) Filed: Jul. 3, 2002

(30) Foreign Application Priority Data
Jul. 4, 2001 (IT) MI2001A001413

(51) Int. Cl. D21H 25/18; A61L 2/16; A61L 2/18

(52) U.S. Cl. 422/1

(57) ABSTRACT

A method for the preservation of paper products, comprising the deacidification of the paper by using diazo derivatives is described.

### FIELD OF THE INVENTION

The present invention concerns a new method for the preservation of paper products which comprises the deacidification of the paper material with the diazo derivatives of general formula (I) hereafter reported.

### STATE OF THE ART

It is universally acknowledged that one of the causes of the too rapid deterioration of cultural materials on paper is the presence of acidity in the material.

In modern paper, acidity is usually caused during the manufacturing stage in the paper factory; however, acidity can often be found even in papers or books that are made from acid-free paper, as it comes from some types of ink for manuscripts, that was widely used in the past.

Experts agree that in order to prolong the life of books and documents that are stored in libraries and archives (according to the experts from three to five times as much) it is necessary to eliminate the acidity from the materials, by using a technique that in the specialised environment is known as “deacidification”. Obviously, in order to avoid the errors committed in the past, new documents and books to be stored should be made with acid-free paper (UNI n. 10332—Paper for documents. Requirements for the maximum duration and durability and UNI n. 10333—Paper for documents. Requirements for duration).

In the Italian public libraries there are currently 30 million books; an equal amount of paper documents are kept in public archives.

From fragmentary surveys carried out in some Italian preservation environments, in agreement with similar research carried out abroad on a wider scale, it has been found that 20-30% of library and archive materials are now so fragile that they cannot be made available for free consultation; the risk of further damage would be too high. Alongside this relatively low percentage however, it has been found that 60-80% of preserved books and documents need to be deacidified or in some way stabilised; otherwise, it would only be a matter of time before all the acid material would become fragile, and no longer consultable.

In view of what above said, it is evident that, in order to protect the Italian book and document heritage, it is necessary to be able to intervene with mass deacidification techniques, or in any case with stabilisation techniques that would slow down deterioration; these would be techniques that allow the entire heritage to be restored in a time span of no more than ten, fifteen years. In Italy, unfortunately, deacidification is currently carried out using solely manual techniques that allow only a few hundred interventions each year on books and archive documents.

The use in Italy of other types of stabilisation techniques is not known to the Applicant. On an international scale, studies on mass deacidification techniques have been ongoing for about 35 years. During this time, about 15 or 20 different intervention techniques have been proposed but none of these is widely diffused as yet. Such a high number of proposals make one realise that there are still many problems to be solved. In a few cases, these are technical problems concerning the safety of the material, and also the safety of the people working on the interventions; in other cases there are doubts as to the effectiveness of the proposed technique or on the durability of the deacidification; or, further still, in many cases, the secondary effects on the materials have been found to be unacceptable.

It is therefore much felt the need for developed and improved techniques for the deacidification of paper products.

### SUMMARY

The Applicant has found a new method for the preservation of paper products, comprising deacidification of paper by using as deacidifying agent with at least one compound of general formula (I) hereafter reported.

The method according to the present invention may be applied to any type of paper and has the following advantages:

unlike most of the known methods, no preliminary dehydration phase for the samples to be treated, with a consequent reduction in costs for a possible industrial process;

after treatment, the pH values remain over 8.5-9.0 in all samples. Even in the case of badly deteriorated papers, that have an initial pH of 2-3 units, the present method guarantees a suitable neutralisation;

homogeneous distribution of the deacidifying agent over all the sample, assisted by the high impregnation capacity of the solvent;

the solvent is easy to remove as it has a low boiling point and low evaporation heat, with consequent reduction in costs for a possible industrial process;

protection, as a result of the present method, from atmospheric acid pollutants (SO₂ and NO₂);

long-term protection effect;

the organic solvent used cannot dissolve any water-soluble substances (colorants, pigments, inks, etc).

Subject of the present invention is therefore a method for the deacidification of paper comprising a deacidification step wherein at least one diazo derivative of general formula (I) is used as deacidifying agent

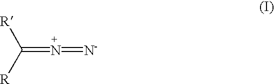

wherein R′ is chosen from H and methyl, and R is the group

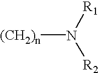

where n=1, 2, 3, 4, 5; and R₁ and R₂, equal to one another, are chosen from methyl and ethyl, or R₁ and R₂, taken together, form with N a piperidine ring or a 4-morpholine ring.

### BRIEF DESCRIPTION OF THE DRAWINGS

FIG. 1 shows the pH value of paper samples from a book dating from 1954 before the deacidification treatment according to the present method (dotted line) and the pH trend vs. days after the treatment (continuous line) as described in Example 1.

### DETAILED DESCRIPTION OF THE INVENTION

According to a particular embodiment of the invention, the present method comprises the following steps:

A) pre-treatment step, consisting of a preliminary and rough removal of any dust or foreign matter from the surface of the paper sheet, using compressed air jets;

B) deacidification step wherein at least one diazo derivative of general formula (I) as defined above, is used as deacidifying agent;

C) washing step, possibly repeated, of the paper coming from step B), and removal of the washing solvent;

D) possible conditioning step.

The pH of the material can be measured using the surface measurement method ( TAPPI T 529 om-99 method).

For pH measurement the cold extraction method (TAPPI T 509 om-96 method) and the hot extraction method (TAPPI T 435 om-96 method) can also be used; these techniques are however, damaging, and therefore cannot be used on paper of historical-documentary interest, but only on samples that are specifically prepared for laboratory tests.

The deacidification step B) of the present method is preferably carried out by completely immersing the paper sample in a special glass container, varying in size according to the amount of material to be treated, and which contains an ethereal solution of the compound of formula (I), freshly prepared and kept in the dark until that moment at a temperature of ≦−180° C.

According to a preferred embodiment, the ratio between the sample weight and the solution volume is about 10 g / 200 ml, with a concentration of the compound of formula (I) that ranges between 0.1 and 0.5 M, depending on the state of the paper to be treated (more concentrated for highly oxidised and acid paper and less concentrated for slightly deteriorated papers).

The container is then closed though not sealed, to avoid a dangerous increase in pressure inside it, and should be preferably kept at 4° C. for a period of 24 to 96 hours (depending on the state of the original material). After this time, the system should be returned to room temperature and the paper is extracted.

The preferred deacidifying agent according to the invention is 2-(1-piperidine) diazoethane, i.e. the formula (I) compound in which R is H, R₁ and R₂, taken together, form with N a piperidine ring, and n is 1.

The diazo derivatives of formula (I) used in the deacidification method of the invention can be prepared by the process that is the subject of the co-pending patent application in the name of the Applicant.

According to a particular embodiment of the present method, the deacidifying agent of formula (I) is applied as steam, by creating a specific chamber in which a propelled vacuum is applied, that is able to create a saturated atmosphere in a short time.

In step C) of the present method the removal of the washing solvent is preferably carried out by drying the paper washed material under strong aspiration at room temperature on a glass surface for about 6 hours. The removal of the solvent can be speeded up via the application of a pressure that is lower than atmospheric pressure (vacuum).

The preferred washing solvent to be used is diethyl ether. Paper treated in this way can then undergo a conditioning step D), for example via storage for 1 week in a insulated container (T 23° C., 50% R.H.) in order to restore the normal content of water in the paper.

To obtain the positive results shown below, it is usually sufficient to apply only one cycle of treatment according to the present method. However, it may be useful to repeat the entire cycle of treatment twice or more.

The following examples are given to provide non-limiting illustrations of the present invention.

### EXAMPLE 1

### Deacidification of paper samples from a book dating from 1954

Preliminary measurements of pH were taken on a number of samples belonging to the book in question, in order to be able to provide an average pH of the whole volume.

After these measurements were taken, using the surface measurement method, and with the cold extraction and hot extraction methods, the entire book was given the average pH value of 4-4.2.

A) The surface of the samples were given a preliminary and rough clean (using compressed air jets) to remove dust and/or residues from the sheet's surface.

B) 20 samples (squares of paper about 4×4 cm) of the average weight of 0.15 g each (for a total of 3 g), were completely immersed in a glass weighing bottle (Ø100 mm, h 50 mm) containing 100 ml of an ethereal solution of 2-(1-piperidine) diazoethane (0.3 M) freshly prepared and kept in a dark place until that moment at a temperature ≦−18° C.

The weighing bottle was closed, but not sealed to avoid a dangerous increase in pressure inside, and kept at 4° C. for 72 hours, then it was gradually returned at room temperature and the samples were removed from it.

C) The paper samples were washed repeatedly with diethyl ether to remove any residue of non reacted 2-(1-piperidine)diazoethane.

After washing, the samples were left to dry under strong aspiration at room temperature on a glass surface for about 6 hours.

D) Finally, after the solvent was completely removed, the samples were conditioned for 1 week in an insulated box (T 23° C., 50% R.H.).

The pH of these samples was measured over 3 months to check the effectiveness of the method.

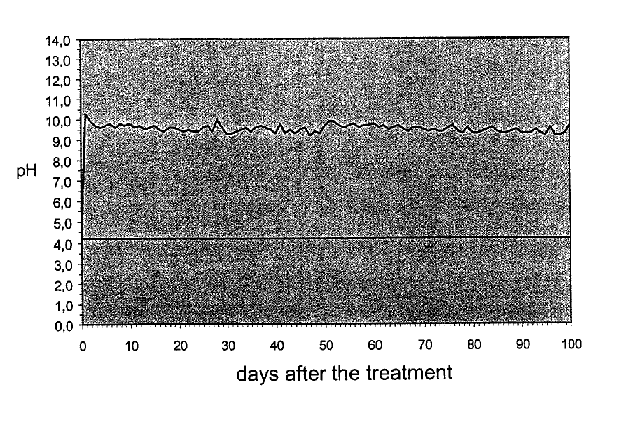

FIG. 1 shows the pH trend before treatment (dotted line) and after treatment (continuous line).

### EXAMPLE 2

### Deacidification of various paper substrates

In order to evaluate the effectiveness, the present method of deacidification was applied to a wide heterogenic range of paper under-layers. Several different paper samples were used for this purpose:

SAMPLE 1) Whatman n°1 chr grade paper, pre-treated with various deteriorating agents (NalO₄, NaOCl, H₂SO₄, HClO₂) in order to create a considerable number of acid groups than can be neutralised with 2-(1-piperidine)diazoethane;

SAMPLE 2) Whatman n°1 chr grade paper artificially aged (T 80° C., 65% R.H.) for 3 months;

SAMPLE 3) paper from old books dating from the 1950s, in a considerably deteriorated state showing a natural acidity (pH=3.5-4).

These samples were subjected to the deacidification method of the invention in the conditions already described above in Example 1, using 2-(1-piperidine) diazoethane as the deacidifying agent.

In all cases, an increase in the pH by at least 4 units was found, going from definitely acid values before deacidification (pH ≡4-5) to clearly alkaline values after treatment (pH≡8-9). The pH trend of the various samples was monitored over a period of time and the alkalinity persisted after 3 months of natural aging (T 23° C., 50% R.H.).

In Table 1 below, for all the types of paper tested the pH values before the treatment according to the deacidifying method of the invention, and the pH values measured after fixed time intervals are reported:

TABLE 1

| | pH before | pH after | pH after | pH after | pH after |
|---|---|---|---|---|---|
| sample | treatment | 1 day | 1 month | 2 months | 3 months |
| 1-NalO₄ | 4.5 | 9.0 | 8.8 | 9.0 | 8.9 |
| 1-NaOCl | 5.0 | 8.5 | 8.7 | 8.5 | 8.5 |
| 1-HClO₂ | 3.5 | 9.5 | 9.3 | 9.2 | 9.0 |
| 1-H₂SO₄ | 3.0 | 9.2 | 9.3 | 8.9 | 9.1 |
| 2 | 5.2 | 9.4 | 9.2 | 9.3 | 9.0 |
| 3 | 4.2 | 10.3 | 10.0 | 9.7 | 9.4 |

As a result of the experiments described above, it was found that using diazo derivatives of formula (I), according to the present method, as deacidifying agents, in particular 2-(1-piperidine)diazoethane, the pH value is increased to more than satisfactory alkaline values for the complete neutralisation of the treated paper samples and these pH values remain practically stable in time.

Also, no changes in colour were found in the period following treatment (yellowing), nor any side effects such as the appearance of stains or spreading of ink.

No unpleasant odours were formed.

1. Method for the deacidification of paper comprising a deacidification step wherein at least one diazo derivative of general formula (I) is used as deacidifying agent 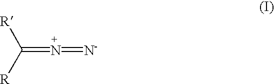
wherein R′ is chosen from H and methyl, and R is the group 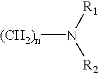
where n=1, 2, 3, 4, 5; and R₁ and R₂, equal to one another, are chosen from methyl and ethyl, or R₁ and R₂, taken together, form with N a piperidine ring or a 4-morpholine ring.

2. The method according to claim 1, comprising the following steps:
A) pre-treatment step, comprising a preliminary and rough removal of any dust or foreign matter from the surface of the paper sheet, by means of compressed air jets;
B) deacidification step wherein at least one diazo derivative of general formula (I) as described in claim 1, is used as deacidifying agent;
C) washing step, possibly repeated, of the paper coming from step B), and removal of the washing solvent;
D) possible conditioning step.

3. The method according to claim 2, wherein the said deacidification step B) is carried out by complete immersion of the paper to be treated in a container containing an ethereal solution of the diazo derivative of formula (I) as described in claim 1.

4. The method according to claim 3, wherein the ratio between the weight of the paper to be treated and the volume of the said ethereal solution is about 10 g/ 200 ml, and the concentration of the diazo derivative (I) in the ethereal solution ranges between 0.1 and 0.5 M.

5. The method according to claim 3, wherein the paper to be treated is maintained at a temperature of 4° C. for a period of time from 24 to 96 hours, and then returned to room temperature before extracting the paper.

6. The method according to claim 2, wherein said deacidification step B) is carried out by the application of the diazo derivative of formula (I) as steam onto the paper to be treated, placed inside a vacuum chamber.

7. The method according to claim 2, wherein the said washing solvent in step C) is diethyl ether.

8. The method according to claim 2, wherein the said removal of the washing solvent is carried out by drying the samples under aspiration at room temperature for 6 hours.

9. The method according to claim 2, wherein said conditioning step D) is carried out by storing the paper coming from step C) for 1 week in an insulated container (T 23° C., 50% R.H.).

10. The method according to claim 1, wherein the said diazo derivative is the compound of formula (I) in which R′ is H, n is 1, and R₁ and R₂, taken together, form with N a piperidine ring.

11. Paper deacidificated according to a method comprising a deacidification step wherein at least one diazo derivative of general formula (I) as described in claim 1, is used as deacidifying agent.
